(19) United States
(12) Patent Application Publication
(10) Pub. No.: US 2017/0266184 A1
(43) Pub. Date: Sep. 21, 2017

(54) NON ADDICTIVE MORPHINE PLUS BUSPIRONE COMBINATION FOR THE TREATMENT OF PAIN

(71) Applicants: Darakhshan Jabeen Haleem, Karachi (PK); Shazia Nawaz, Karachi (PK); Tabinda Salman, Karachi (PK); Muhammad Iqbal Choudhary, Karachi (PK)

(72) Inventors: Darakhshan Jabeen Haleem, Karachi (PK); Shazia Nawaz, Karachi (PK); Tabinda Salman, Karachi (PK); Muhammad Iqbal Choudhary, Karachi (PK)

(21) Appl. No.: 15/070,722

(22) Filed: Mar. 15, 2016

(51) Int. Cl. A61K 31/506; A61K 31/485

(52) U.S. Cl. CPC A61K 31/506; A61K 31/485

(57) ABSTRACT

The present invention is a development of a morphine plus buspirone combination (7.5 mg/kg morphine plus 1-2 mg/kg buspirone) used as a non-addictive treatment for killing severe pain. This combination is more effective than morphine alone for the treatment of pain and is more than 90% less addictive.

### BACKGROUND OF THE INVENTION

Morphine, the main pharmacologically active alkaloid of opium poppy, is a potent analgesic and clinically used to treat moderate to severe pain, which includes acute surgical pain, cancer associated pain and chronic non-cancer pain. However, long term use of morphine and other opiates produces tolerance in its therapeutic effects [Dumas, E. O. & Pollack, G. M. 2008, Opioid tolerance development: A pharmacokinetics/pharmacodynamic perspective. AAPS J. 10(4), 537-551]. Patients treated with morphine like drugs for killing pain show dependence and also drug overuse [Morgan, M. M. & Christie, M. J. 2011, Analysis of opioid efficacy, tolerance, addiction and dependence from cell culture to human. Br J Pharmacol. 164(4), 1322-1334] which further limits the clinical utility of these drugs.

Nociception is modulated by multisynaptic descending pathways that include both opioidergic and serotonergic mechanisms [Duman, E. N., Kesim, M., Kadioglu, M., Yaris, E., Kalyoncu, N. I., & Erciyes, N. 2004, Possible Involvement of opioidergic and serotonergic mechanisms in anti-nociceptive effect of paroxetine in acute pain. J Pharmacol Sci. 94, 161-165]. It has been reported that acute morphine administration enhances 5-hydroxytryptamine (5-HT, serotonin) turnover as evidenced by an increase in its synthesis, release and metabolism. Some studies show that subcutaneous injection of 8-hydroxy-N,N-dipropyl-2-aminotetralin (8-OH-DPAT) as 5-HT-1A receptor agonist produces an analgesic effect in the formalin model of tonic nociceptive pain [Bardin, L., Tarayre, J. P., Koek, W., & Colpaert, F. C. 2001, In the formalin model of the tonic nociceptive pain, 8-OH-DPAT produces 5-HT-1A receptor-mediated, behaviorally specific analgesia. Eur J Pharmacol. 421, 109-14].

As the serotonergic systems can modulate pain, and drugs acting on 5-HT systems e.g., selective serotonin reuptake inhibitors (SSRIs) are also often used in combination with opioids for treating pain. Thus fluoxetine potentiates the anti-nociceptive effects of morphine in rats and rhesus monkeys [Hynes, M. D., Lochner, M. A., Bemis, K. G., & Hymson, D. L. 1985, Fluoxetine, a selective inhibitor of serotonin uptake, potentiates morphine analgesia without altering its discriminative stimulus properties or affinity for opioid receptors. Life Sci. 36, 2317-2323]. Moreover, the 5-HT releaser fenfluramine and the SSRI fluoxetine increase analgesic effects of morphine in humans [Coda, B. A., Hill, H. F., Schaffer, R. L., Luger, T. J., Jacobson, R. C., & Chapman, C. R. 1993, Enhancement of morphine analgesia by fenfluramine in subjects receiving tailored opioid infusions. Pain 52, 85-91; Erjavec, M. K., Coda, B. A., Nguyen, Q., Donaldson, G., Risler, L., & Shen, D. D. 2000, Morphine-fluoxetine interactions in healthy volunteers: analgesia and side effects. J Clin Pharmacol. 40, 1286-1295].

It is clear that agonist activity at certain 5-HT receptor subtypes can modulate nociception. For example, the 5-HT-1A receptor agonist 8-OH-DPAT has anti-nociceptive effects in rats [Crisp, T., Stafinsky, J. L., Spanos, L. J., Uram, M., Perni, V. C., & Donepudi, H. B. 1991, Analgesic effects of serotonin and receptor-selective serotonin agonists in the rat spinal cord. Gen Pharmacol. 22, 247-251] and the SSRI clomipramine enhances the anti-nociceptive effects of opioids in monkeys [Banks, M. L., Rice, K. C., and Negus, S. S. 2010, Anti-nociceptive interactions between mu-opioid receptor agonists and the serotonin uptake inhibitor clomipramine in rhesus monkeys: Role of mu agonist efficacy. J. Pharmacol Exp Ther. 335, 497-505].

It is also reported that combined and continuous administrations of morphine and 5-HT-1A receptor agonists inhibits development of tolerance to morphine analgesia in trigeminal neuropathic pain [Deseure, K. R., Adriaensen, H. F., & Colpaert, F. C. 2004, Effects of the combined continuous administration of morphine and the high-efficacy 5-HT-1A agonist, F 13640 in a rat model of trigeminal neuropathic pain. Eur J Pain. 8, 547-54].

Buspirone, an azaspirodecanedione derivative is clinically recommended for the treatment of patients with generalized anxiety disorder [Rickels, K. 1990, Buspirone in clinical practice. J Clin Psychiatry. 51 (Suppl.), 51-54]. It has partial affinity for 5-HT-1A receptor as agonist and dopamine D2 receptors as an antagonist [Gobert, A., Rivet, J. M., Cistarelli, L., Melon, C., & Millan, M. J. 1999, Buspirone modulates basal and fluoxetine-stimulated dialysate levels of dopamine, noradrenaline and serotonin in the frontal cortex of freely moving rats: activation of serotonin-1A receptors and blockade of alpha2-adrenergic receptors underlie its actions. Neuroscience. 93, 1251-1262]. Previously, we have shown that addiction to apomorphine is not produced when buspirone and apomorphine are co-administered to rats [Haleem, D. J., Ikram, H., & Haleem, M. A. 2014, Inhibition of apomorphine-induced conditioned place preference in rats co-injected with buspirone: relationship with serotonin and dopamine in the striatum. Brain Res. 1586, 73-82]. We therefore suggested a role of 5-HT-1A receptor in addiction [Haleem, D. J. 2013, Extending therapeutic use of psychostimulants: focus on serotonin-1A receptor. Prog Neuropsychopharmacol Biol Psychiatry. 46, 170-180]. This study was conducted to determine if buspirone can inhibit morphine abuse and psychosis and whether analgesic effects of morphine are modulated or remain intact by this co-administration.

### BRIEF SUMMARY OF THE INVENTION

Morphine, the main pharmacologically active alkaloid of opium poppy, is a potent analgesic and is widely used to treat chronic pain; however its utility is hindered by the development of tolerance, dependence and addiction when the drug is used repeatedly. Serotonin has been shown to have a role in the rewarding/reinforcing effects of drug of abuse. 5-HT agonist/antagonists with selectivity towards various receptors can modulate reinforcing effects and sensitization by drug of abuse. The present study was designed to improve therapeutic use of morphine, both from the perspective of decreasing unwanted effects and from the perspective of enhancing therapeutic effects.

A method is provided for inhibiting addictive potential of morphine while improving pain relieving efficacy of morphine. Here we show that buspirone if co-administered with morphine could improve therapeutic use of morphine. Addiction to morphine is more than 90% reduced and its analgesic effects are potentiated in buspirone co-injected animals. Moreover, psychosis like effect is not produced with this combination of morphine plus buspirone.

### BRIEF DESCRIPTION OF THE SEVERAL VIEWS OF THE DRAWINGS:

FIG. 1 A shows results of conditioned place preference (CPP) test used to monitor addictive effects of morphine (7.5 mg/kg) in the absence or presence of buspirone. Data analyzed by three-way ANOVA repeated measure shows a significant effect of morphine (F=131.5 df1,30 p<0.01), buspirone (F=22 df2,30 p<0.01) and a significant interaction between morphine and buspirone (F=23.9 df2,30 p<0.01). Repeated measure (day) effects (F=80.1 df1,30 p<0.01), day×morphine (F=121.4 df1,30 p<0.01), day×buspirone (F=20.1 df2,30 p<0.01) and day×buspirone×morphine (F=34.8 df2,30 p<0.01) interactions are also significant.

Post hoc analysis by Tukey's test shows that pre-conditioning values of time spent in the drug paired compartment are comparable in the six groups. Post-conditioning values of time spent in the drug paired compartment are much higher in morphine treated animals suggesting repeated use of morphine is addictive. Co-administration of buspirone at doses of 1 mg or 2 mg/kg significantly attenuates morphine-induced increases in post conditioning values of time spent in drug paired compartment suggesting inhibition in the addictive effects of morphine in buspirone co-treated animals.

FIG. 1B depicts calculated percentage reinforcement values. T-test shows that morphine at doses of 7.5 mg/kg is reinforcing. The reinforcing effects do not occur if buspirone (1 mg or 2 mg/kg) is co-administered.

FIG. 2A depicts the effects of morphine, buspirone and their co-administration on motor behavior and percentage psychosis like effects. Three way ANOVA (repeated measure design) shows significant effect of morphine (F=21.76 df1,30 p<0.01), buspirone (F=70.7 df2,30 p<0.01) and a significant interaction between morphine and buspirone (F=19.8 df2,30 p<0.01). Repeated measure (day) effects (F=17 df5,30 p<0.01), day×morphine (F=5.05 df5,30 p<0.01), day×buspirone (F=28.2 df10,30 p<0.01) and day×buspirone×morphine (F=24.6 df10,30 p<0.01) interactions are also significant.

Post hoc analysis by Tukey's test shows that 1st and 2nd injection of morphine produces a decrease in motor activity. The decreases do not occur following 3rd to 6th administration of morphine on day 6, 8, 10 and 12, suggesting that tolerance in motor depressant effect of morphine is produced after 2nd administration. Tolerance in motor depressant effects of morphine is not produced in animals co-treated with buspirone at doses of 1 mg/kg or 2 mg/kg. Calculated percentage psychosis like effects of morphine are also blocked in animals co-treated with buspirone.

FIG. 2B shows that morphine produced psychosis like effects which are attenuated by co-administration of 2 mg/kg buspirone and blocked by co-administration of 1 mg/kg buspirone

FIG. 3 depicts effect of different doses of morphine on heat-induced nociception in rats tested on hot plate. One-way ANOVA shows significant morphine effect on latency to lick onset (F=57.4 df3,20 p<0.01) and number of licks (F=54.8 df3,20 p<0.01). Post-hoc analysis shows an increase in latency time in paw licking at all three doses of morphine as compared to saline treated animals. The increase is greater at 7.5 mg/kg than 5 mg/kg morphine. Number of licks is reduced in morphine than saline treated animals at all three doses.

FIG. 4 depicts the effects of morphine, buspirone and their co-administration on heat-induced nociception. Two way ANOVA (repeated measure design) shows significant effect of morphine (F=8.850 df1,30 p<0.01) and buspirone (F=43.180 df2,30 p<0.01) on latency to first lick. Interaction between morphine and buspirone (F=2.528 df2,30 p>0.05) is not significant. Post hoc analysis by Tukey's test shows that latency time to first lick is reduced in repeated morphine treated animals suggesting that repeated administration of morphine produces hyperalgesia. The hyperalgesic effects do not occur in morphine plus buspirone co treated animals.

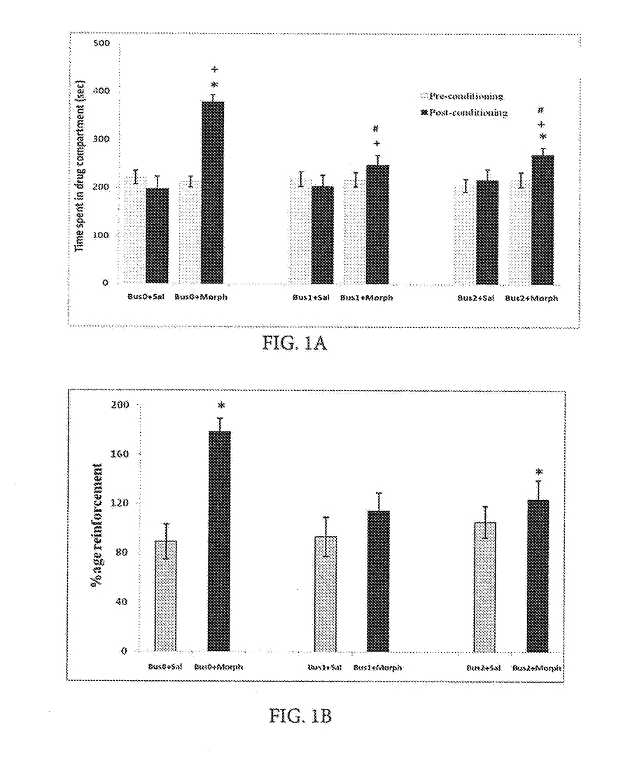

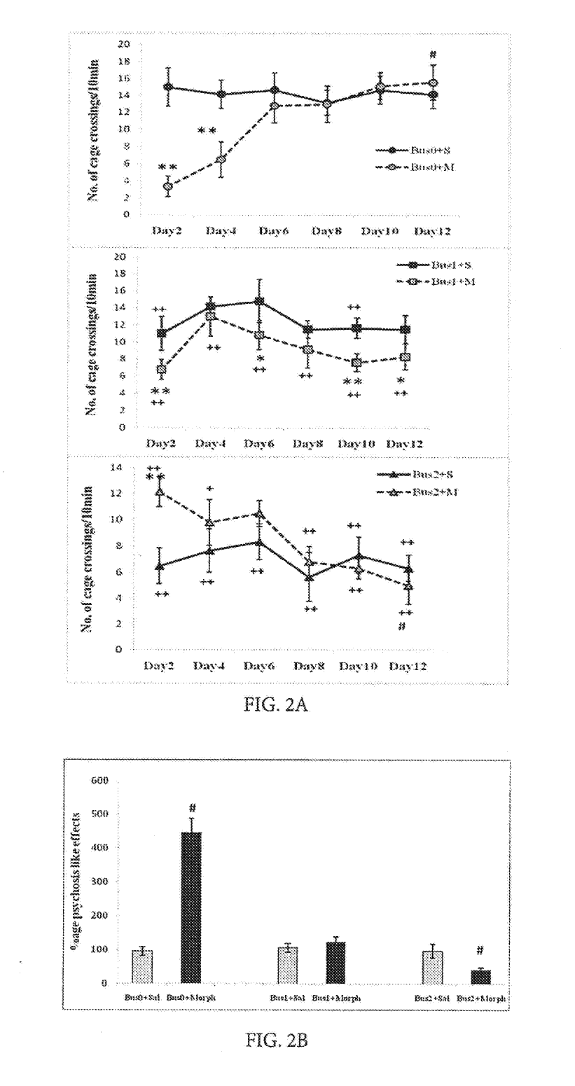

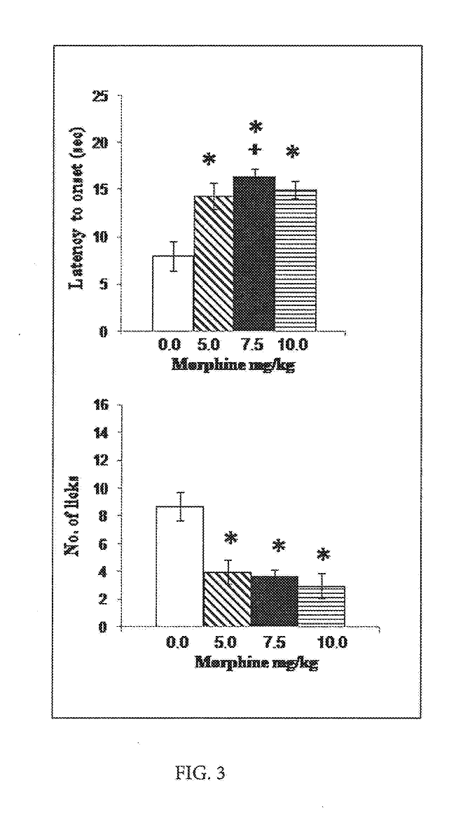

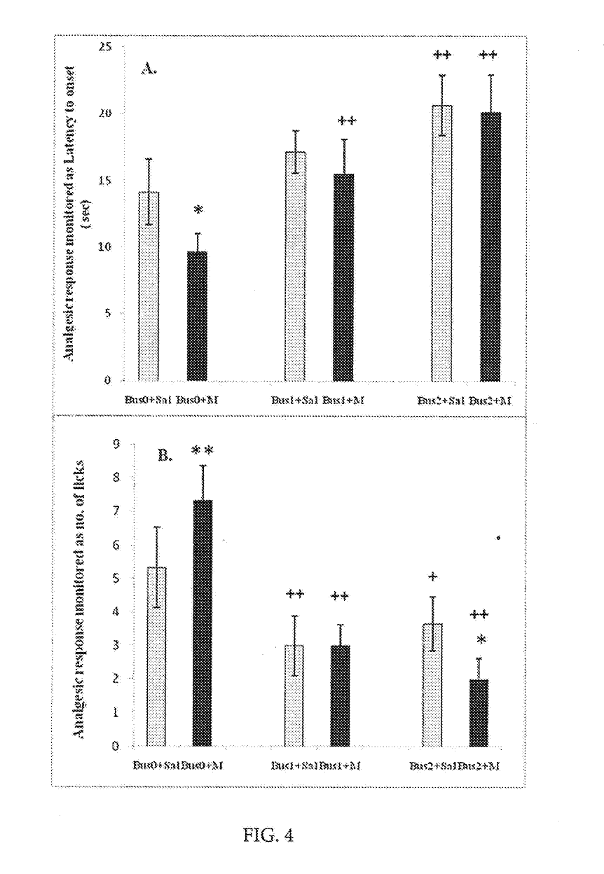

Two way ANOVA (repeated measure design) performed on number of licks shows significant effect of buspirone (F=58.472 df2,30 p<0.01) and significant interaction between morphine and buspirone (F=12.639 df2,30 p<0.01) on number of licks. Effect of morphine (F=0.139 df1,30 p>0.05) are not significant. Post hoc analysis by Tukey's test shows that number of licks is higher in repeated morphine treated animals, suggesting hyperalgesia in repeated morphine treated animals and its inhibition in animals treated with morphine plus buspirone.

### DETAILED DESCRIPTION OF THE INVENTION

Experiment:

Co-administration of morphine with buspirone to study effects on morphine induced addiction, sensitization and analgesic effects

Apparatus for Conditioned Place Preference

A three compartment conditioned place preference (CPP) apparatus with an unbiased design was used to monitor drug-induced reinforcement. The apparatus was made up of transparent plastic Perspex. The compartments were separated by sliding guillotine doors. The transparent middle (shuttle) compartment (14×26×26 cm) had a smooth floor and no stripes.

The end (preference) compartments (26×26×26 cm each) provided distinct contexts, with one compartment having black horizontal stripes on side walls, the other compartment had vertical black stripes. In this apparatus, animals showed no consistent preference for either compartment, which supported our unbiased CPP paradigm. The experiment was conducted in three distinct phases: pre-conditioning, conditioning and post-conditioning.

Pre-Conditioning Test

A pre-conditioning test ensured that the animals did not have a preference for any of the compartments. An animal introduced in the CPP apparatus from the middle compartment and guillotine doors were raised to open. The animal was allowed to explore the entire apparatus for 10 min. Time spent in each compartment was recorded.

Conditioning Phase

This phase started one day after pre-conditioning phase, animals were randomly assigned to six groups, each containing six animals: (i) buspirone 0 mg/kg+saline, (ii) buspirone 0 mg/kg+morphine, (iii) buspirone 1 mg/kg+saline, (iv) buspirone 1 mg/kg+morphine, (v) buspirone 2 mg/kg+saline and (vi) buspirone 2 mg/kg+morphine injected animals. Over next 12 days (day 1 to day 12) animals went through conditioning (one session per day) in which they were confined to either the horizontal or vertical stripe compartment by raising the respective guillotine door. On day 1, 3, 5, 7, 9 and 11 animals of all groups were injected with saline (1 ml/kg) and placed immediately in the assigned ‘Non-Drug’ compartment for 30 min. After which the animals were kept in their home cages. On every other day, i.e. day 2, 4, 6, 8, 10, and 12 control and test animals were injected with saline, buspirone and/or morphine as assigned above. Immediately after injection the animals were placed in the ‘Drug’ compartment for 30 min. The animals picked from the drug compartment were placed back in their home cages.

Post-Conditioning Test

The test was carried out on day 13, one day after the last conditioning session, in a drug free state. Each animal was tested only once. As in the pre-conditioning phase, the guillotine doors were removed and rat was given access to the entire apparatus for 10 min. Time spent in ‘Drug’ assigned compartment was monitored.

Motor Sensitization

Motor behavior was also monitored during conditioning phase. Animals confined to a compartment were moving across the compartment. Activity scores were counted as number of cage crossings for 10 min starting 5 min post injection.

Hot Plate Test

After post-conditioning the challenge dose of morphine (7.5 mg/kg) was given. The hot plate test was conducted 30 min after challenge dose to measure difference in heat-induced nociception in the six groups. The latency (either jumping or hind-paw licking) and number of licks were monitored for each animal. The temperature of hot plate was kept 52° C. and cutoff time was 30 sec.

1. (canceled)

2. A method of reducing hyperalgesia to morphine in animals and humans by administering to an animal or human in need thereof morphine at a dose of 7.5 mg/kg in combination with buspirone at a dose of 1-2 mg/kg buspirone.
